(19) United States
(12) Patent Application Publication
(10) Pub. No.: US 2011/0165536 A1
(43) Pub. Date: Jul. 7, 2011

(54) ALVEOLAR RIDGE AUGMENTATION

(75) Inventors: Hadar BETTER, Tel-Aviv (IL); Gidoen Fostick, Givat Shmuel (IL); Yossi Gross, Moshav Mazor (IL)

(73) Assignee: RAINBOW MEDICAL LTD., Herzeliya (IL)

(21) Appl. No.: 12/683,153

(22) Filed: Jan. 6, 2010

(51) Int. Cl. A61C 19/00; A61B 18/18; A61B 17/24; A61C 8/00

(52) U.S. Cl. 433/32; 601/2; 433/173; 433/80

(57) ABSTRACT

Apparatus is provided for use with a gingival periosteum lining a bone. The apparatus comprises a periosteal mesher, which comprises a mesher surface, and a plurality of cutting elements distributed over the mesher surface, which are configured to cut the gingival periosteum to increase flexibility thereof. Other embodiments are also described.

### FIELD OF THE APPLICATION

Some embodiments of present invention relate generally to dental implants and implantation methods, and specifically to ridge augmentation devices and methods.

### BACKGROUND OF THE APPLICATION

Osseointegrated dental implants are typically metallic or ceramic screws that are placed in the jawbone for supporting artificial teeth after the loss of natural teeth. Replacement of the teeth is often a challenging surgical procedure when the remaining alveolar bone has insufficient height or width to support the implant. Before the dental implant is implanted, the mandibular or maxillary alveolar ridge must be reconstructed, in order to provide sufficient height and width.

Guided bone regeneration (GBR) is a reconstruction method that uses a barrier membrane to direct the growth of new bone along the alveolar ridge. The method includes opening the gingival tissue along the length of the alveolar ridge, and placing a regenerative material (such as autogenic, allogeneic, xenogeneic, or synthetic bone graft) against the ridge, and a membrane on the regenerative material. The gingival tissue is sutured to cover the membrane. The membrane maintains the regenerative material in place and prevents the gingival tissue from growing into the regenerative material and interfering with bone regeneration. The regenerative material integrates with the existing alveolar bone, providing the necessary alveolar width to support the implant.

U.S. Pat. No. 7,396,232 to Fromovich et al. describes devices and methods for gradual displacing of the soft tissues covering bones. The gap developing between the bone and the displaced soft tissue will be filled with bone callus as it is in distraction osteogenesis. The devices and methods allow formation of bone in distraction osteogenesis without cutting a segment of the bone. The devices and methods are particularly useful in dental implantology for vertical ridge augmentation by displacing the periosteal tissue and for sinus lift by displacing the Schneiderian membrane. The devices and methods can also regenerate soft tissue between the bone and the displaced soft tissue.

U.S. Pat. No. 6,402,518 to Ashman describes a method and apparatus for augmenting an endentulous alveolar ridge of a patient. The method comprises the steps of (1) making a provisional denture-stent with a hollow space on the underside to account for the width, height and extent of the desired augmentation; (2) making an incision in, and reflecting, the gingiva where the augmentation is desired; (3) inserting bone graft material on the cortical plate; (4) suturing the gingiva; and (5) inserting the provisional stent over the bone graft material.

US Patent Application Publications 2008/0103518 and 2009/0101157 to Karmon describe bioresorbable inflatable devices and tunnel incision tool and methods for treating and enlarging a tissue or an organ or a tube or a vessel or a cavity. The device comprises a hollow expanding pouch made of a resorbable or a perforated material that can be attached to a filling element. The pouch is filled with biocompatible materials, one or more times in an interval of a few days, after the insertion of the device. While the pouch is filled every few days, the tissue expands and the filling material, if it is bioactive, starts to function. The tunnel incision tool comprises a small blade that emerges from the surface of the tool in order to make shallow incisions in the surrounding tissue, therefore enabling easy expansion of the tissue. This device and method can be used, for example, for: horizontal and vertical bone augmentation in the jaws, when the tunnel incision tool is used to make shallow incisions in the periosteum when using the tunnel technique; sinus augmentation, when the device is placed beneath the Schneiderian tissue; vessel widening, when the pouch becomes a stent; or fixating bone fractures.

U.S. Pat. No. 4,755,184 to Silverberg describes an implant for use in bone augmentation. The implant includes a hollow casing made of a resorbable material and a prosthetic filling material for bones contained within the casing. A method for bone augmentation includes the steps of making an incision adjacent to the augmentation site, inserting an implant comprising a hollow casing made of resorbable porous material and filled with a prosthetic filling material for bones, into the augmentation site directed via the incision, and closing the incision.

U.S. Pat. No. 4,798,205 to Bonomo et al. describes a subperiosteal tissue expander for reconstruction of the edentulous atrophied alveolar ridge of the mandible or maxilla. The expander includes an inflatable tube curved into a “C” shape to match the curvature of the human alveolar ridge, having a layer of reinforcement material on one side of the inflatable tube and tabs for attachment of lines at either end of the inflatable tube. Also described is a method for reconstructing the edentulous atrophied alveolar ridge of the maxilla or the mandible by placing a tissue expander subperiosteally along the alveolar ridge of the maxilla or mandible, inflating the expander to create a subperiosteal channel, removing the expander, and filling the subperiosteal channel with a hard material such as hydroxylapatite.

U.S. Pat. No. 6,030,218 to Robinson describes a sub-periosteally implantable prosthesis support structure for a fixed or detachable dental prosthesis, which includes a framework fitted to and generally conforming to the inner and outer contours of the bony ridge structures of a person. The framework is configured to provide a space extending generally normal to the bony ridge structure to an apex to provide space for subsequent bone growth. A plurality of denture support posts are distributed about the framework and depend outwardly from the apex in substantial alignment with the bony ridge structure. During the fabrication of the prosthesis support structure, a bio-compatible fine mesh screen is fixed to and spans, tent-like, the framework to substantially overlay the bone structure and the space provided for subsequent bone growth. After the support structure has been implanted, the growth of bone into the space and around the support structure is promoted to osseointegrate the support structure with the person's bony ridge, thus providing a secure foundation for a denture or fixed dental prosthesis configured for detachable or fixed coupling with the denture support posts. The support structure may be made, partly or wholly, from either non-resorbable material, such as titanium stock and mesh, or from a resorbable material such as Vicryl™.

U.S. Pat. No. 7,357,637 to Liechtung describes an appliance fabricated to improve the appearance of a patient's smile. The appliance includes a plurality of simulated teeth. The interior surfaces of each of the teeth closely fits and conforms to the surface of a patient's real teeth while the outer surfaces of each of the simulated teeth has an ideal surface configuration. The dental appliance provides the patient with the appearance of a perfect set of teeth and an ideal smile without a need to alter the dental structure of the patient's teeth.

Skin graft meshers are designed to incise and “mesh” a piece of skin prior to grafting in order to allow the piece to expand and cover a larger area than its donor site area. For example, Brennen Medical, LLC (Saint Paul, Minn., USA) distributes a skin graft mesher for conserving donor site tissue in cases of extensive skin loss.

The following references may be of interest:
U.S. Pat. No. 5,695,338 to Robert
U.S. Pat. No. 5,858,082 to Cruz et al.
U.S. Pat. No. 6,050,819 to Robinson
U.S. Pat. No. 6,063,094 to Rosenberg
U.S. Pat. No. 6,126,662 to Carmichael et al.
U.S. Pat. No. 6,148,232 to Avrahami
U.S. Pat. No. 6,409,764 to White et al.
U.S. Pat. No. 7,364,430 to Kitamura et al.
U.S. Pat. No. 7,537,616 to Branch et al.
US Patent Application Publication 2007/0055285 to Osorio et al.
US Patent Application Publication 2007/0088436 to Parsons et al.
US Patent Application Publication 2007/0093899 to Dutoit et al.
US Patent Application Publication 2007/0207186 to Scanlon et al.
US Patent Application Publication 2009/0163918 to Levy et al.

### SUMMARY OF EMBODIMENTS

In some embodiments of the present invention, surgical tools and methods are provided for cutting the gingival periosteum lining the maxillary or mandibular alveolar ridge. Such cutting increases the flexibility of the periosteum, thereby facilitating stretching of the periosteum to encompass a larger volume and accommodate the introduction of regenerative material between the periosteum and the ridge. A periosteal mesher is provided, which includes a surface comprising a plurality of cutting elements distributed over the surface. After the periosteal mesher is inserted between the gingiva and the bone, the cutting elements are used to cut the periosteum. For some applications, the cutting elements comprise energy-applying elements, which are activated to apply energy to the periosteum, thereby cutting the periosteum. Alternatively or additionally, the cutting elements comprise mechanical cutting elements, such as blades.

In some embodiments of the present invention, an external gingival cap is provided for augmenting an atrophied portion of an alveolar ridge. The gingival cap is shaped such that a surface thereof that faces the alveolar ridge defines a desired geometry of the atrophied portion of the alveolar ridge. After affixing the gingival cap to the ridge, a bone regenerative material is introduced into a cavity between the gingiva and the atrophied ridge. The regenerative material assumes the defined geometry upon integrating with the bone of the ridge. After integration, or after introduction of the regenerative material but before integration, a dental implant is implanted in the augmented ridge. A dental appliance, such as a crown, is coupled to the dental implant.

In addition to defining the desired geometry, the gingival cap protects the regenerative material from moderate forces applied to the ridge during normal use of the mouth while the regenerative material integrates with the ridge. Augmentation using the gingival cap is generally simpler to perform than conventional augmentation procedures, and provides at least as much augmentation. For some applications, portions of the gingival cap are configured to be affixed to respective teeth of the subject. For some applications, the gingival cap comprises or is shaped so as to define one or more simulated teeth, such that the gingival cap additional serves an aesthetic function.

Some embodiments of the present invention provide a dental stent implant and minimally-invasive techniques for alveolar ridge augmentation. A small incision is made in the gingiva, and the gingiva is loosened from the bone of the ridge. Via the incision, the stent implant is introduced in a collapsed state between the gingiva and the bone. The stent implant is fixed to the bone and expanded, typically using one or more expandable chambers, such as balloons. For some applications, the implant is fixed to the bone before it is expanded, while for other application, the stent is first expanded and then fixed to the bone. Expansion of the stent implant separates the gingiva from the bone along the length of the stent implant, creating a volume within the stent implant and along the sides thereof. A bone regenerative material is introduced into and around the stent implant. The gingival incision is sutured. With the aid of the support provided by the stent, the regenerative material integrates with the existing alveolar bone, providing the necessary alveolar width and/or height to support a dental implant. After integration, or after introduction of the regenerative material but before integration, a dental implant is implanted in the augmented ridge. A dental appliance, such as a crown, is coupled to the dental implant.

In some embodiments of the present invention, the stent implant provides a plurality of portions disposed along a length of the stent implant. The portions are configured to be differentially expandable. Differentially expanding the portions allows the expanded shape of the stent implant to create and define a desired geometry of space alongside the ridge that is to be augmented. This allows the augmented space to better conform to the natural shape of the ridge, which is often geometrically complex.

For some applications, a separate expandable chamber, such as a balloon, is provided for each stent portion, and the expandable chamber are inflated to expand the respective portions. Alternatively, a single expandable chamber is provided which is shaped so as to define a plurality of portions therealong having respective cross-sectional areas when the expandable chamber is inflated. The expandable chamber is placed within the stent implant such that the expandable chamber portions are generally aligned with respective portions of the stent implant.

Further alternatively, a single expandable chamber is provided, which initially extends to a vicinity of a distal end of the stent implant. The expandable chamber is inflated until the distal-most portion of the stent implant is expanded to a desired cross-sectional area. The expandable chamber is then withdrawn proximally until it is positioned within the proximally adjacent stent portion. This selective inflation and withdrawal is repeated until all of the stent portions have been expanded.

There is therefore provided, in accordance with an embodiment of the present invention, apparatus for use with a gingival periosteum lining a bone, the apparatus including a periosteal mesher, which includes:

a mesher surface; and

a plurality of cutting elements distributed over the mesher surface, which are configured to cut the gingival periosteum to increase flexibility thereof.

For some applications, the cutting elements include energy-applying elements, which are configured to cut the gingival periosteum by applying energy to the gingival periosteum. For some applications, the energy-applying elements include electrodes. For some applications, the apparatus further including a radiofrequency (RF) generator, which is configured to drive a RF current through the electrodes. For some applications, the electrodes protrude from the mesher surface.

For some applications, the energy-applying elements include ultrasound transducers.

For some applications, the mesher includes between 1 and 30 energy-applying elements.

Alternatively or additionally, the cutting elements include mechanical cutting elements, which are configured to mechanically cut the gingival periosteum.

For some applications, the cutting elements are configured to create cuts that extend through only a portion of a thickness of the gingival periosteum, and not entirely through the gingival periosteum.

For some applications, at least a portion of the cutting elements are arranged parallel to one another on the mesher surface.

For some applications, the apparatus further including a kit, which includes the mesher and a bone regenerative material.

There is further provided, in accordance with an embodiment of the present invention, a method including:

inserting, between an alveolar ridge and gingiva lining the ridge, a periosteal mesher that includes a plurality of cutting elements distributed over a surface of the mesher; and

increasing flexibility of a gingival periosteum by cutting the gingival periosteum using the cutting elements.

For some applications, the cutting elements include energy-applying elements, and cutting the gingival periosteum includes activating the energy-applying elements to apply energy to the gingival periosteum. For some applications, the energy-applying elements include electrodes, and activating includes driving a current through the electrodes. For some applications, driving the current includes driving a radiofrequency (RF) current through the electrodes. For some applications, the energy-applying elements include ultrasound transducers, and activating includes activating the ultrasound transducers to generate ultrasound waves.

For some applications, increasing the flexibility of the periosteum includes facilitating stretching of the periosteum to encompass a larger volume between the periosteum and the ridge, and further including introducing a regenerative material between the periosteum and the ridge. For some applications, introducing includes introducing the regenerative material via a delivery tube that includes a valve.

For some applications, increasing the flexibility of the periosteum includes stretching the cut gingival periosteum.

For some applications, cutting includes creating cuts that extend through only a portion of a thickness of the gingival periosteum, and not entirely through the gingival periosteum.

For some applications, the cutting elements include mechanical cutting elements, and cutting the gingival periosteum includes mechanically cutting the gingival periosteum using the mechanical elements. For some applications, mechanically cutting includes creating cuts that extend through only a portion of a thickness of the gingival periosteum, and not entirely through the gingival periosteum.

For some applications, the method further includes, before inserting, identifying that the gingiva is recessed; and after cutting the gingival periosteum, stretching the gingiva toward crowns of one or more teeth of the ridge.

For some applications, inserting includes separating the gingiva from the ridge.

There is still further provided, in accordance with an embodiment of the present invention, apparatus for augmenting an atrophied portion of an alveolar ridge of a subject, the apparatus including an external gingival cap, which includes:

at least one cap portion, which is shaped so as to define a surface that faces the alveolar ridge and defines a desired geometry of the atrophied portion of the ridge; and

one or more ridge-coupling portions, which are configured to be removably coupled to the alveolar ridge, and to hold the cap portion in place.

For some applications, the apparatus further including a kit, which includes the external gingival cap and a bone regenerative material. For some applications, the kit further includes a delivery tube that includes a valve, which delivery tube is configured to deliver the bone regenerative material.

For some applications, the external gingival cap further includes one or more simulated teeth, coupled to the at least one cap portion.

For some applications, the one or more ridge-coupling portions include one or more tooth-coupling portions, which are configured to be removably coupled to respective ones of teeth of the ridge, in order to couple the external gingival cap to the alveolar ridge. For some applications, the cap portion is shaped so as to define one or more simulated teeth. For some applications, the one or more tooth-coupling portions are shaped so as to define one or more simulated teeth. For some applications, the cap portion and the one or more tooth-coupling portions are shaped so as to define a plurality of simulated teeth.

There is additionally provided, in accordance with an embodiment of the present invention, a method for augmenting an alveolar ridge of a subject, including:

separating gingiva from an atrophied portion of the alveolar ridge to create a cavity between the gingiva and the atrophied portion of the ridge;

affixing an external gingival cap to the ridge such that a surface of a cap portion of the gingival cap that faces the ridge defines a desired geometry of the atrophied portion of the ridge;

before or after affixing the cap, introducing a regenerative material into the cavity;

waiting for the regenerative material to at least partially integrate with the bone of the ridge, such that the ridge assumes the desired geometry; and

after waiting, removing the external gingival cap from the ridge.

For some applications, the method further includes, before separating the gingiva, making an incision through a site of the gingiva in a vicinity of the atrophied portion of the ridge.

For some applications, introducing includes introducing an amount of regenerative material sufficient to expand the cavity and push the gingiva against the surface of the gingival cap.

For some applications, affixing includes affixing one or more tooth-coupling portions of the gingival cap to respective teeth of the subject. For some applications, the cap portion is shaped so as to define one or more simulated teeth. For some applications, the one or more tooth-coupling portions are shaped so as to define one or more simulated teeth. For some applications, the cap portion and the one or more tooth-coupling portions are shaped so as to define a plurality of simulated teeth.

For some applications, introducing includes introducing the regenerative material via a delivery tube that includes a valve.

There is yet additionally provided, in accordance with an embodiment of the present invention, apparatus including a dental augmentation kit, which includes:

a stent implant, which is shaped so as to provide a plurality of differentially expandable portions arranged along the length; and

a bone regenerative material.

For some applications, the stent implant is shaped so as to provide at least three differentially expandable portions.

There is also provided, in accordance with an embodiment of the present invention, a method including:

making an incision through gingiva of an alveolar ridge;

via the incision, introducing a stent implant in a collapsed state between the gingiva and bone of the ridge, the stent implant shaped so as to provide a plurality of differentially expandable portions arranged along the length;

differentially expanding the portions to respective expanded states; and

thereafter, introducing a regenerative material into the stent implant.

For some applications, the stent implant is shaped so as to provide at least three differentially expandable portions, and differentially expanding includes differentially expanding the at least three portions.

The present invention will be more fully understood from the following detailed description of embodiments thereof, taken together with the drawings, in which:

### BRIEF DESCRIPTION OF THE DRAWINGS

FIGS. 1A-B are schematic illustrations of a dental stent implant, in accordance with an embodiment of the present invention;

FIGS. 2A-B are schematic illustrations of a plurality of expandable chambers for expanding the stent implant of FIGS. 1A-B, in accordance with an application of the present invention;

FIGS. 3A-B are schematic illustrations of an expandable chamber for expanding the stent implant of FIGS. 1A-B, in accordance with an application of the present invention;

FIGS. 4A-H are schematic illustrations of a stent expansion method using an expandable chamber, in accordance with an application of the present invention;

FIGS. 5A-E are schematic cross-sectional illustrations of a portion of the stent implant of FIGS. 1A-B, in accordance with respective applications of the present invention;

FIGS. 6A-F are schematic illustrations of several steps of a minimally-invasive alveolar ridge augmentation procedure that uses the stent implant of FIGS. 1A-B, in accordance with an embodiment of the present invention;

FIG. 7 is a schematic illustration of a periosteal mesher, in accordance with an embodiment of the present invention;

FIGS. 8A-B are schematic illustrations of exemplary configurations of electrodes on a surface of the periosteal mesher of FIG. 7, in accordance with respective applications of the present invention;

FIG. 9 is a schematic illustration of a portion of a procedure performed using the periosteal mesher of FIG. 7, in accordance with an application of the present invention;

FIGS. 10A-B are schematic illustrations of an external gingival cap, in accordance with respective embodiments of the present invention;

FIGS. 11A-F are schematic illustrations of several steps of a minimally-invasive alveolar ridge augmentation procedure that uses the external gingival cap of FIGS. 10A-B, in accordance with an application of the present invention;

FIG. 12 is a schematic illustration of another external gingival cap, in accordance with an application of the present invention;

FIGS. 13A-F are schematic illustrations of several steps of a minimally-invasive alveolar ridge augmentation procedure that uses the external gingival cap of FIG. 12, in accordance with an application of the present invention; and

FIG. 14 is a schematic illustration a delivery tube comprising a valve, for delivering regenerative material, in accordance with an application of the present invention.

### DETAILED DESCRIPTION OF EMBODIMENTS

FIGS. 1A-B are schematic illustrations of a dental stent implant 10, in accordance with an embodiment of the present invention. Stent implant 10 provides a plurality of portions 12 disposed along a length of the stent implant, such as at least two (e.g., exactly two), at least three (e.g., exactly three), or at least four (e.g., exactly four) portions (three portions 12A, 12B, and 12C are shown in the figures). Stent implant 10 typically comprises a plurality of interconnected struts 14, as is well known in the cardiovascular stent art. The stent implant comprises a biocompatible material, such as plastic or metal, such as titanium or Nitinol. For some applications, the material is biodegradable. The phrase “stent implant,” as used in the present application including the claims, should be understood broadly to include any elongated structure that provides structural support to tissue, and that can assume collapsed and expanded states.

For some applications, stent implant 10 is tubular and is configured to expand radially outward, such as is well known in the cardiac stent art. For other applications, the stent implant is rolled like a scroll in its compressed state, and expands by unrolling (configuration not shown).

Stent implant 10 typically has a length of between 5 and 40 mm, and, when expanded, typically surrounds a volume of between 0.25 and 5 ml.

For some applications, struts 14 of stent implant 10 are configured to provide different mesh densities on different portions of the stent implant. For example, the mesh may be highly porous on the side of the stent implant that faces the ridge, and tightly knit on the periosteal side. The porous side aids with the biological connection with the bone, while the tightly knit side provides structure to the stent.

Portions 12 are configured to be differentially expandable. When stent implant 10 assumes a collapsed state, as shown in FIG. 1A, all of portions 12 typically have approximately the same cross-sectional area and shape. When stent implant 10 assumes an expanded state, as shown in FIG. 1B, portions 12 assume respective cross-sectional areas, at least some of which differ from one another. In addition, for some applications, when the stent implant assumes the expanded state, the portions optionally assume respective, different cross-sectional shapes.

Differentially expanding portions 12 allows the expanded shape of stent implant 10 to create and define a desired geometry of space alongside the ridge that is to be augmented. This allows the augmented space to better conform to the natural shape of the ridge, which is often geometrically complex.

For some applications, portions 12 of stent implant 10 are configured during manufacture of the stent to assume predefined respective shapes and/or cross-sectional areas upon expansion. For other applications, the respective expanded shapes and/or cross-sectional areas are established during the dental procedure, by differentially expanding the respective portions of the stent implant, such as using one or more expandable chambers, such as balloons, as described hereinbelow.

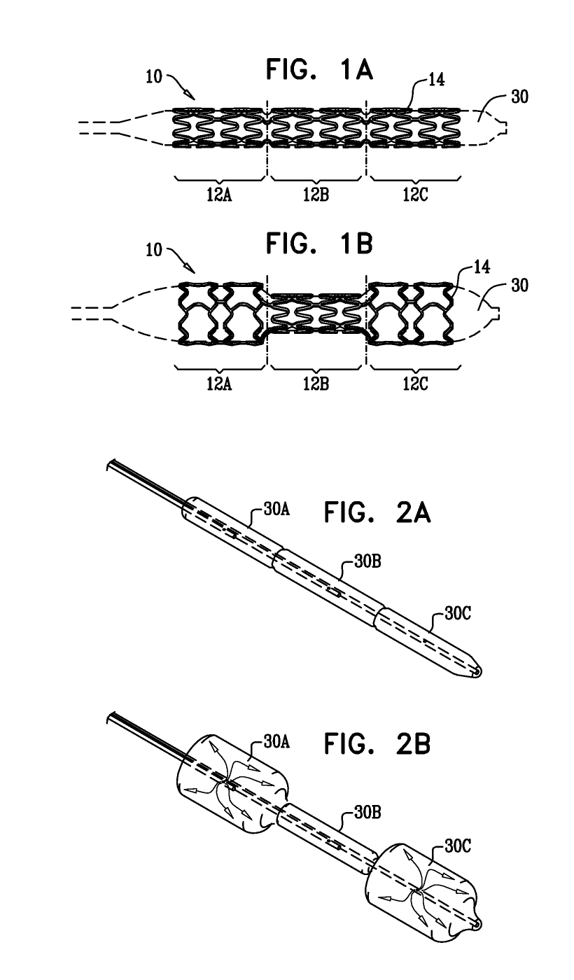

Reference is made to FIGS. 2A-B, which are schematic illustrations of a plurality of expandable chambers 30, such as balloons, for expanding stent implant 10, in accordance with an application of the present invention. In this application, a separate expandable chamber 30 is provided for each stent portion, and the expandable chambers are inflated to expand the respective portions. For example, as shown in FIGS. 2A-B, expandable chambers 30A, 30B, and 30C may be provided for expanding stent portions 12A, 12B, and 12C, respectively, of stent implant 10 shown in FIGS. 1A-B.

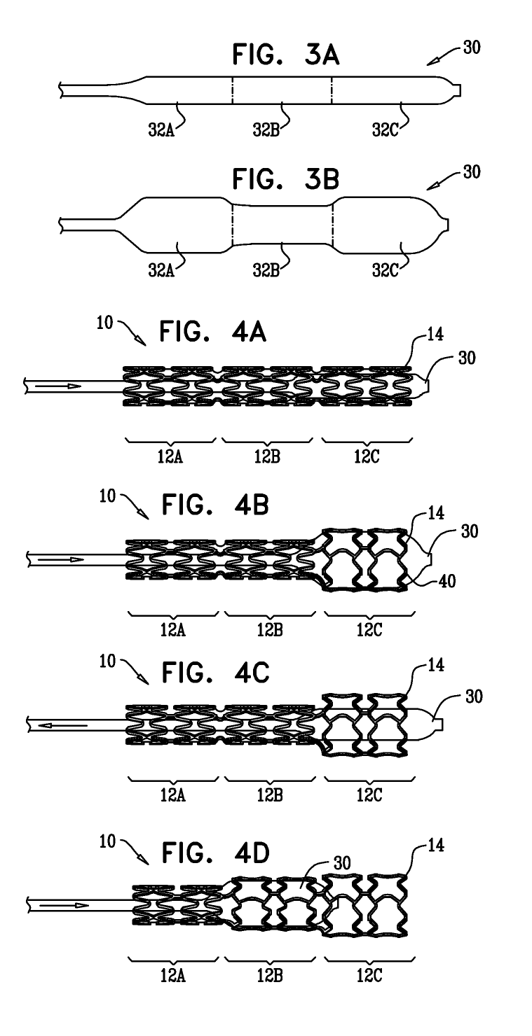

Reference is made to FIGS. 3A-B, which are schematic illustrations of expandable chamber 30 for expanding stent implant 10, in accordance with an application of the present invention. In this application, an expandable chamber 30 (typically, a single expandable chamber 30) is provided which is shaped so as to define a plurality of portions 32 therealong having respective cross-sectional areas when the expandable chamber is inflated. The expandable chamber is placed within the stent implant such that the expandable chamber portions are generally aligned with respective portions of the stent implant. For example, as shown in FIGS. 3A-B, expandable chamber portions 32A, 32B, and 32C may be provided for expanding stent portions 12A, 12B, and 12C, respectively, of stent implant 10 shown in FIGS. 1A-B.

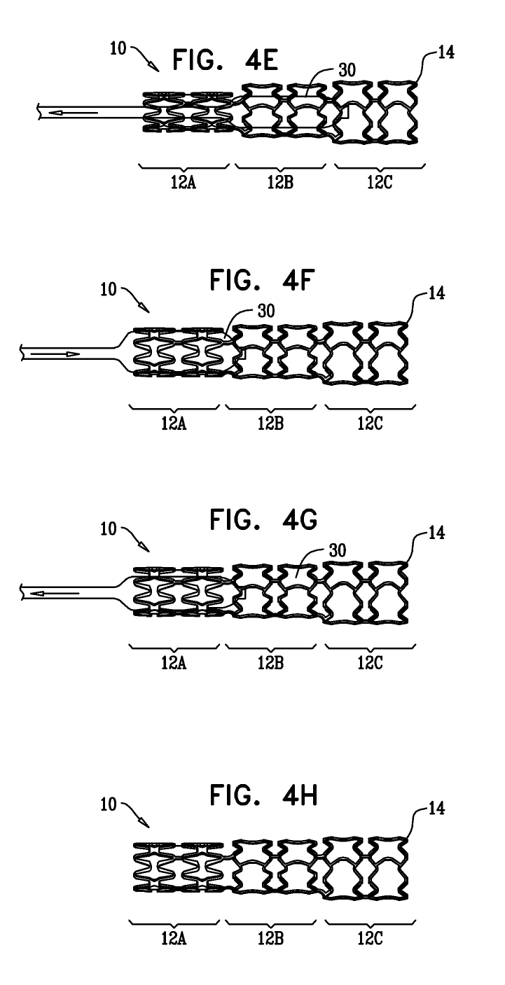

Reference is made to FIGS. 4A-H, which are schematic illustrations of a stent expansion method using expandable chamber 30, in accordance with an application of the present invention. In this application, an expandable chamber 30 (typically, a single expandable chamber 30) is provided, which initially extends to a vicinity of a distal end 40 of stent implant 10, as shown in FIG. 4A. Expandable chamber 30 is inflated until a first, distal-most portion 12C of stent implant 10 is expanded to a desired cross-sectional area, as shown in FIG. 4B. The expandable chamber is then at least partially deflated, as shown in FIG. 4C. The expandable chamber is withdrawn proximally until it is positioned within a second, proximally adjacent stent portion 12B, and, as shown in FIG. 4D, is again inflated until this second stent portion 12B is expanded to a desired cross-sectional area. This selective inflation and withdrawal is repeated, as shown in FIGS. 4E-G, until all of the stent portions have been expanded, as shown in FIG. 4H.

For some applications, expandable chambers 30 are provided that combine the techniques described hereinabove with reference to FIGS. 2A-B, FIGS. 3A-B, and/or FIGS. 4A-H.

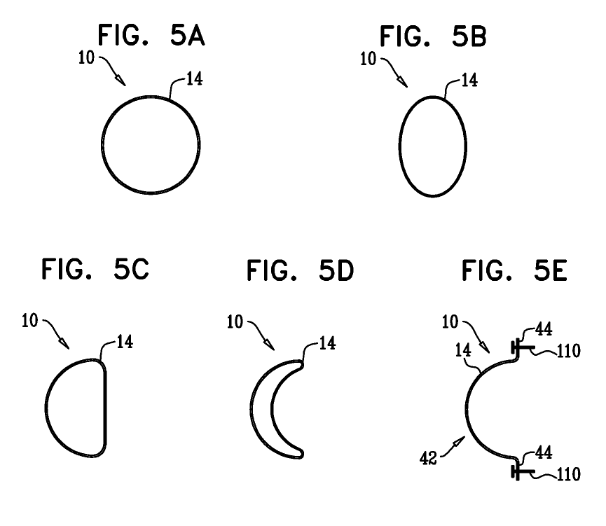

Reference is made to FIGS. 5A-E, which are schematic cross-sectional illustrations of a portion of stent implant 10, in accordance with respective applications of the present invention. Stent implant 10 may have various cross-sectional shapes when in its expanded position. For example, the cross-sectional shape may be circular (as shown in FIG. 5A), elliptical (as shown in FIG. 5B), generally D-shaped (as shown in FIG. 5C), or generally crescent-shaped (as shown in FIG. 5D). If D-shaped, the stent implant is oriented so that the flatter side faces the ridge. If crescent-shaped, the stent implant is oriented so that the concave side of the crescent faces the ridge, and the convex side faces the gingiva.

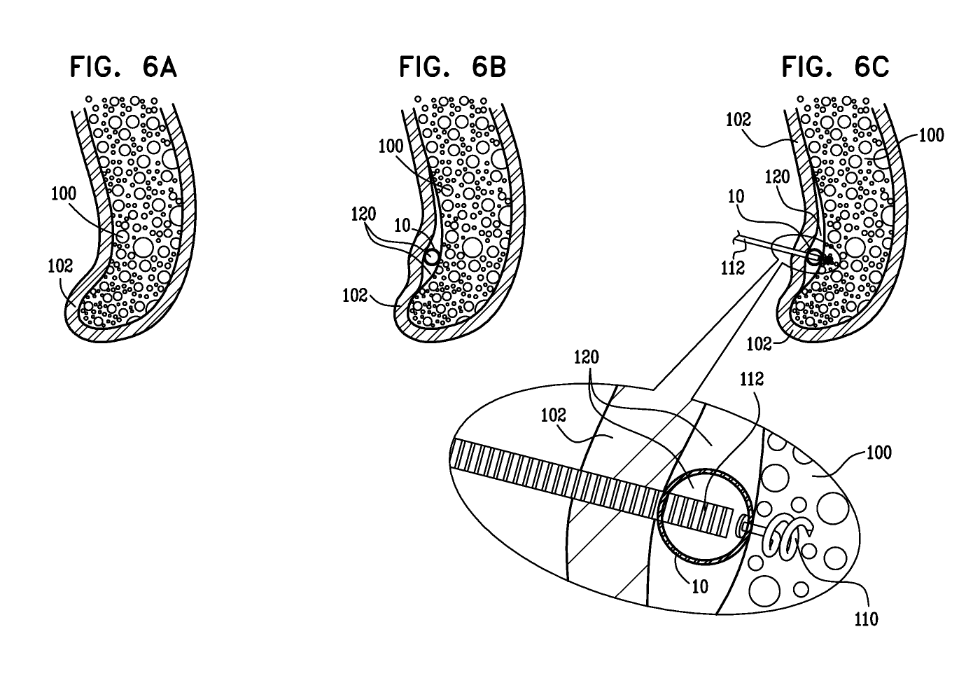

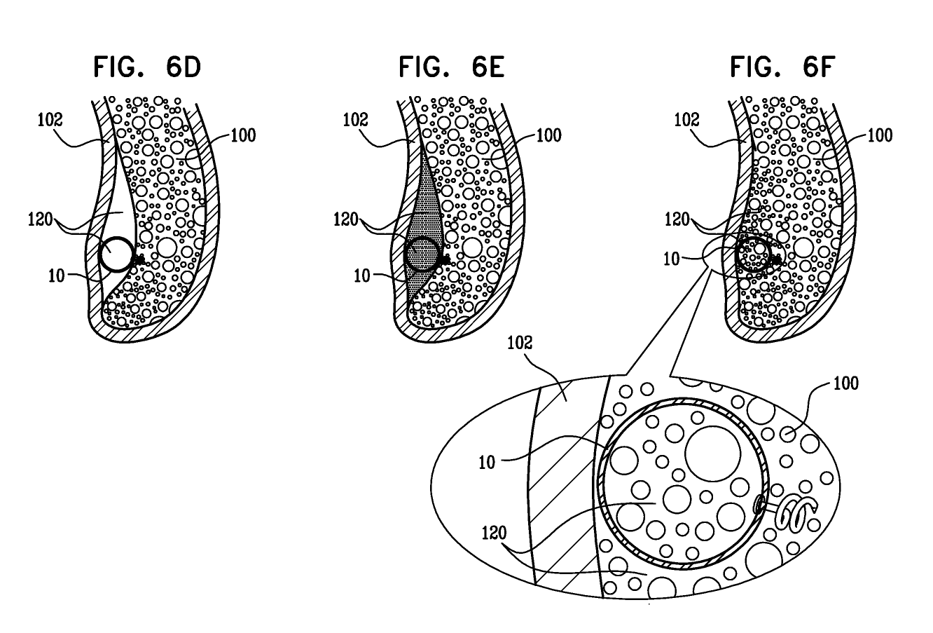

For some applications, stent implant 10 is entirely open on one side, such as shown in FIG. 5E. In these applications, the stent implant typically includes a curved portion 42 and two wings 44. The stent implant is oriented so that wings 44 and the concave side of curved portion 42 face the ridge, and the convex side of curved portion 42 faces the gingiva. Typically, fasteners 110, described hereinbelow with reference to FIG. 6C, are passed through wings 44.

As mentioned above, different portions 12 of the stent implant may have different cross-sectional shapes.

Reference is now made to FIGS. 6A-F, which are schematic cross-sectional illustrations of several steps of a minimally-invasive alveolar ridge augmentation procedure that uses stent implant 10, in accordance with an application of the present invention. The procedure is typically employed when a patient's endentulous or partially endentulous maxillary or mandibular alveolar ridge 100 lacks sufficient bone width and/or height to support a dental implant, as shown in FIG. 6A. For example, the procedure may be employed for implanting an implant to replace the upper incisors, lower incisors, upper canines, lower canines, upper premolars, lower premolars, upper molars, or lower molars.

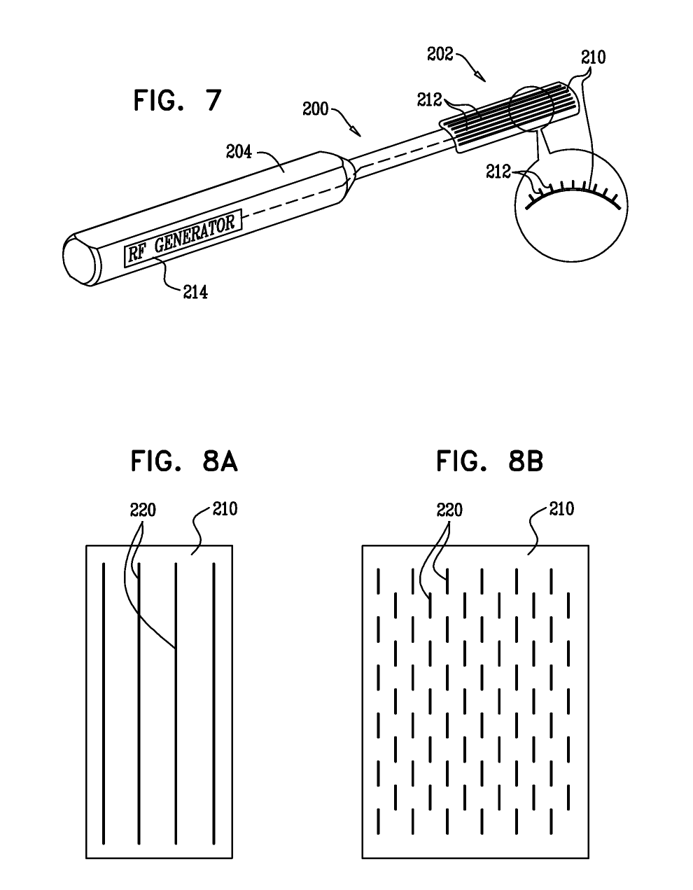

A surgeon begins the procedure by preparing the oral facial region, and administering a local anesthetic. A small incision is made through a site of gingiva 102, and a portion of the gingiva is loosened from the bone of ridge 100, thereby creating a small cavity 120 between the gingival portion and the bone. The gingiva is typically loosened by tunneling using a periosteal elevator (e.g., a Molt 9 periosteal elevator), as is known in the art, or using radiofrequency energy, ultrasound energy, a water jet, or a laser. Alternatively or additionally, the surgeon uses periosteal mesher 200, which is described hereinbelow with reference to FIGS. 7-9.

Via the incision, stent implant 10 is introduced in a collapsed state between the portion of gingiva 102 and the bone of ridge 100, as shown in FIG. 6B. For some applications, an introducer tool is used to introduce the stent implant. For example, the tool may be placed within the stent implant.

As shown in FIG. 6C, stent implant 10 is fixed to the bone of ridge 100. For some applications, the stent implant is fixed to the bone while the stent implant is still in its collapsed state. Alternatively, the stent is first expanded, as described hereinbelow with reference to FIG. 6D, and subsequently fixed to the bone. For some applications, the stent implant is fixed to the bone using one or more (e.g., three or four) fasteners 110, which may comprise, for example, screws, tacks (e.g., distributed by Salvin Dental Specialties, Inc. (Charlotte, N.C., USA)), or nails. For example, each of the fasteners may have length of between 3 and 5 mm. For some applications, the fasteners comprise a metal, such as titanium, while for other applications the fasteners comprises a biodegradable material, such as PLA-PGA.

The fasteners may be pre-positioned in the collapsed stent before the stent is introduced between the gingiva and the bone. Optionally, a biodegradable structure inside the stent holds the fasteners until they are fixed to the bone. Optionally, the fasteners are integral to the stent. Alternatively, the fasteners may be introduced during the procedure, either via the proximal end of the stent implant, or via respective openings in the wall of the stent implant that faces the gingiva. For some applications, the openings are labeled with a dark marking visible through external tissue, such as a circle around each of the openings. For some applications, a fastening tool 112, such as a screwdriver or a mallet, is used to fasten the fasteners to the bone. For some applications, fastening tool 112 is shaped so as to define a sharp tip that punctures and penetrates the gingiva and engages each of the fasteners (typically one at a time). For other applications, an expandable chamber, such as a balloon, is inflated within the stent implant to apply a sideways force on the fasteners. For some applications, the stent is shaped so as to define one or more (typically two or more) external wings, and the fasteners couple the wings to the bone, as described hereinabove with reference to FIG. 5E.

The stent implant is expanded, as shown in FIG. 6D, typically using one or more expandable chambers 30, such as balloons, such as described hereinabove with reference to FIGS. 2A-B, FIGS. 3A-B, and/or FIGS. 4A-H. For some applications, the one or more expandable chambers are positioned within the stent implant before the stent is introduced between the gingiva and the bone (for example, if the stent implant has wings 44, as described hereinabove with reference to FIG. 5E). For other applications, the one or more expandable chambers are introduced into the stent implant after the stent implant has been introduced between the gingiva and the bone, and, optionally, after the stent implant has been fixed to the bone. Expansion of stent implant 10 separates gingiva 102 from the bone of ridge 100 along the length of the stent implant, thereby enlarging cavity 120 within the stent implant and along the sides thereof.

The expansion of the stent implant occurs quickly in a single session during the surgical procedure. Typically, the expansion occurs over a time period that is less than five minutes, such as less than one minute, or less than one second, e.g., generally instantaneously.

For some applications, as described above, the stent implant assumes its compressed state when relaxed. For other applications, the stent implant assumes its expanded state when relaxed. For these applications, the stent implant is initially mechanically held in the compressed state, and, once fixed to the bone, is released and allowed to assume the relaxed expanded state. For example, the stent implant may be rolled like a scroll in its compressed state, and may expand by unrolling.

As shown in FIG. 6E, a bone regenerative material 130 is introduced into cavity 120. Typically, regenerative material 130 is introduced only into stent implant 10, and spreads by itself into the rest of the cavity around the stent implant. The gingival incision is sutured. For some applications, the regenerative material comprises a fluid, a liquid, a gel, a particulate solid, or a suspension (i.e., a particulate solid suspended in a liquid). For some applications, the pressure and/or volume of the regenerative material is monitored as it is introduced, such as using a pressure gauge. Such monitoring may help prevent damaging, such as by tearing, the tissue around the cavity.

As shown in FIG. 6F, with the aid of the support provided by the stent, regenerative material 130 integrates with the existing alveolar bone, providing the necessary alveolar width and/or height to support a dental implant. After integration, or after introduction of the regenerative material but before integration, a dental implant is implanted in the augmented ridge (not shown). A dental appliance, such as a crown, is coupled to the dental implant (not shown).

The surgeon generally selects an orientation of stent implant 10 that is appropriate for the particular patient's surgery. For some patients, the surgeon aligns the stent generally along alveolar ridge 100, as shown in FIGS. 6A-F. For other patients, the surgeon aligns stent implant 10 in a direction generally perpendicular to alveolar ridge 100 (i.e., generally along a superior-inferior axis of the body), or in another direction, as appropriate.

Reference is now made to FIG. 7, which is a schematic illustration of a periosteal mesher 200, in accordance with an embodiment of the present invention. Periosteal mesher 200 is used to cut gingival periosteum lining the atrophied maxillary or mandibular alveolar ridge. Such cutting facilitates stretching of the periosteum to accommodate the introduction of bone regenerative material between the periosteum and the ridge. Typically, the cuts extend through only a portion of the thickness of the periosteum, and not entirely through the periosteum.

Periosteal mesher 200 comprises a mesher portion 202 coupled to a handle 204. Mesher portion 202 is shaped so as to define a surface 210 that comprises a plurality of cutting elements 212 distributed thereover.

For some applications, cutting elements 212 comprise energy-applying elements, such as between 1 and 30 elements. For some applications, the energy-applying elements comprise:
electrodes, as shown in FIG. 7. For example, each of the electrodes may have a length of between 1 and 30 mm. The electrodes may protrude slightly from surface 210, as shown in the cross-sectional blow-up in FIG. 7, in order to provide good contact with the periosteum and/or slice through the periosteum (in some cases, the height of the protrusions from surface 210 defines the depth of the cuts made through the periosteum). For example, the electrodes may protrude from the surface by up to 2 mm. Alternatively, the electrodes are flush with surface 210, i.e., do not protrude therefrom. For some applications, a radiofrequency (RF) generator 214 is provided, which is configured to drive a RF current through the electrodes. The RF generator may be housed in mesher 200 (such as in handle 204), or may be external to the mesher (configuration not shown). Typically, the electrodes comprise non-insulated elongated conductors, such as wires;
ultrasound transducers (not shown);
water jets (not shown);
lasers (not shown); and/or
resistive elements, for applying ohmic heating.

Typically, surface 210 has a length of between 1 and 30 mm and a width of between 1 and 20 mm, and/or an area of between 1 and 600 mm2. For some applications, mesher portion 202, other than cutting elements 212, comprises a metal, such as stainless steel.

Reference is made to FIGS. 8A-B, which are schematic illustrations of exemplary configurations of electrodes 220 on surface 210 of mesher 200, in accordance with respective applications of the present invention. In the configuration shown in FIG. 8A, electrodes 220 are arranged in parallel to one another, and generally extend continuously along the surface 210. In the configuration shown in FIG. 8B, the electrodes are arranged in lines parallel to one another, such that electrodes in adjacent lines are at least partially offset from one another. Further alternatively, rather than being aligned in a single direction, portions of the electrodes are aligned in different respective directions, such as at right angles to one other (configuration not shown). Alternatively, the electrodes are arranged in a different configuration.

For some applications, cutting elements 212 of periosteal mesher 200 comprise one or more mechanical cutting elements, such as blades, instead of or in addition to the energy-applying elements.

For some applications, periosteal mesher 200 is used in combination with stent implant 10, external gingival cap 300, external gingival cap 400, and/or the dental procedures described herein. For some applications, a portion of struts 14 of stent implant 10 serve as electrodes 220. For other applications, the mesher is used without the stent implant, external gingival caps, and/or procedures described herein.

For some applications in which the cutting elements comprise elongated conductors, such as wires, the cutting elements are configured to be left in place after cutting the periosteum, and to serve as reinforcement scaffolding. For these application, the cutting elements may be detached from surface 210 and surface 210 may be removed. Alternatively, the cutting elements may remain attached to surface 210, in which case the surface also remains in place permanently.

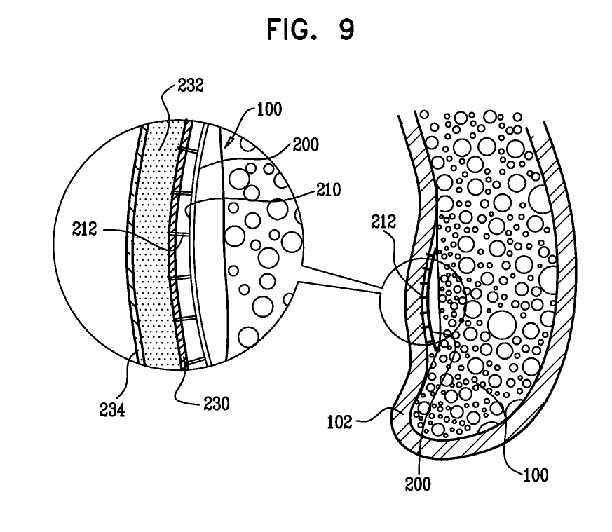

Reference is made to FIG. 9, which is a schematic cross-sectional illustration of a portion of a procedure performed using periosteal mesher 200, in accordance with an application of the present invention. The surgeon begins the procedure by making an incision in the gingiva (not shown). For some applications, a conventional periosteal elevator is used to separate the gingiva from the bone of the ridge (not shown). Alternatively, periosteal mesher 200 is used to separate the gingiva from the bone.

As shown in FIG. 9, periosteal mesher 200 is inserted in the cavity created between gingiva 102 and alveolar bone 100. Cutting elements 212 are used to cut the periosteum. For applications in which cutting elements 212 comprise energy-applying elements, the elements are activated to apply energy to periosteum 230, including a periosteal surface facing the bone, and/or a deeper portion of the periosteum, thereby cutting the periosteum. As shown in the figure, and as well known in the art, gingiva 102 comprises periosteum 230 facing the bone, connective tissue 232, and epithelium 234. For some applications, the surgeon thoroughly meshes the periosteum by advancing and withdrawing the mesher several times, and/or by moving the mesher from side to side several times. This movement may be less necessary when the electrode configuration shown in FIG. 8B is used.

For some applications, cutting elements 212 are coupled to one or more tips of a pivoting instrument, e.g., an instrument similar to a hemostat. For some applications, the cutting elements are provided on both grasping surfaces of the pivoting instrument, while for other applications, the cutting elements are provided only on a single one of the grasping surfaces. For some applications, an elongated incision is made in the gingiva, and gingival flaps are separated from the alveolar ridge. Mesher 200 is applied to both sides of the flaps to mesh the periosteum.

For some applications, mesher 200 is configured to be used during a gingival papilla or muco-gingival surgical procedure for treating gingival recession. In the procedure, the recessed gingiva is reflected from the bone, and mesher 200 creates cuts in the gingival periosteum, which enable increased stretching of the gingiva in a direction perpendicular to the cuts. For example, the mesher may create mesiodistal cuts in the gingival periosteum, or cuts in another direction. After the gingival periosteum is cut, the gingiva are folded back toward the teeth and stretched toward crowns of the teeth. Typically, the gingiva are held stretched by suturing together the gingiva on opposite sides of the teeth. This procedure thus enables the treatment of recessed gingiva without the need for an implant.

For some applications, mesher 200 is used to mesh tissue other than gingival periosteum, such as periosteum elsewhere in the body (e.g., of facial bone), skin, scar tissue, or keloid tissue. For these applications, the mesher may create cuts that partially or completely penetrate the tissue. The mesher may comprise energy-applying elements and/or mechanical cutting elements.

For some applications, a method is provided for aiding in the integration of liquid bone regenerative material. The method comprises introducing, into a cavity in a body of a subject, such as cavity 120 described herein, a bundle of strands of very fine soft metal filaments. The bundle is typically arranged as a compressible scaffold, i.e., a three-dimensional mesh material with a high ratio of overall volume to material volume, to allow bone to grow inside the bundle. For example, the bundle may be similar to conventional steel wool. The bundle typically reduces motion of the bone graft during integration, which motion is caused by ordinary movement of the mouth. The bundle typically provides only minimal mechanical support, and thus cannot resist the application of external forces applied to the gingiva. The high surface area of the bundle may facilitate osseointegration.

For some applications, the bundle is compressible and/or self-expanding. For some applications, the strands of the bundle comprise Nitinol, titanium, and/or a resorbable material such as PLA-PGA or collagen.

A kit for performing this method comprises the bundle of strands and the liquid bone regenerative material. When provided in the kit, the bundle typically has a volume of between 0.25 and 10 ml, and the liquid bone regenerative material typically has a volume of between 0.25 and 10 ml.

For some applications, the surgeon sculpts the bundle to a desired shape to conform to the cavity, before, after, or both before and after introducing the bundle into the cavity. In contrast, the expanded shape of conventional stents is generally fixed, and cannot be readily modified by the surgeon to conform to the shape of a cavity. Furthermore, the mesh of the bundle tends to contain the liquid bone regenerative material, unlike a stent, which typically does not hold a liquid material well.

For some applications, the surgeon uses an introducer to introduce the bundle into the cavity. Withdrawal of the introducer may allow the bundle to expand. For example, the introducer may comprise a tube or tweezers.

For some applications, stent implant 10, described hereinabove with reference to FIGS. 1A-B, comprises the bundle, which is typically positioned within the stent implant. The combined stent implant and bundle provide support that reduces motion of the bone graft during integration. The bundle expands as the stent expands, typically because of the bundle's springiness.

For some applications, proteolytic enzymes are used to dissolve connective tissue 232 and/or the periosteum. These enzymes may be used in combination with mesher 200, or separately.

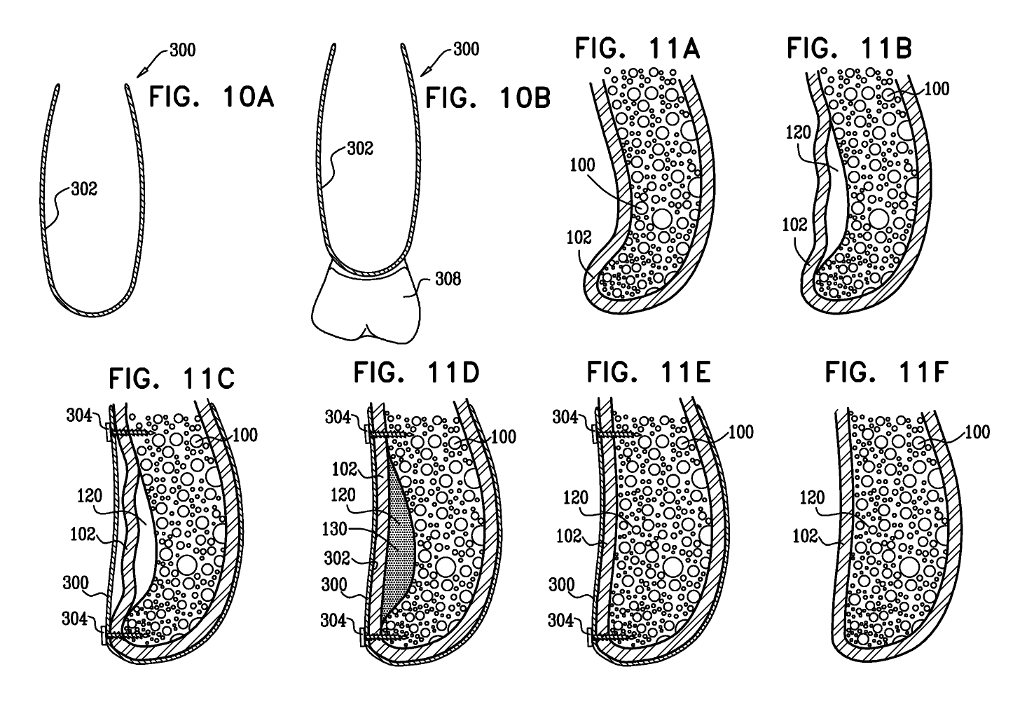

Reference is now made to FIGS. 10A-B, which are schematic cross-sectional illustrations of an external gingival cap 300, in accordance with respective embodiments of the present invention. Gingival cap 300 is used for augmenting an atrophied portion of alveolar ridge 100, such as described hereinbelow with reference to FIGS. 11A-F. Gingival cap 300 is shaped such that a cap portion thereof defines a surface 302 thereof that faces alveolar ridge 100 defines a desired geometry of the atrophied portion of the alveolar ridge. Bone regenerative material introduced into cavity 120 between gingiva 102 and the atrophied ridge assumes the defined geometry upon integrating with the bone of the ridge. In addition to providing the desired geometry, the gingival cap protects the regenerative material from moderate forces applied to the ridge during normal use of the mouth while the regenerative material integrates with the ridge. For some applications, the regenerative material comprises a fluid, a liquid, a gel, a particulate solid, or a suspension (i.e., a particulate solid suspended in a liquid).

Gingival cap 300 typically comprises a generally hard material, such as acetyl resin, methyl methacrylate, or acrylic. Typically, the cap has a thickness of between 0.1 mm and 10 mm.

Reference is made to FIG. 10B. For some applications, gingival cap 300 is shaped so as to define one or more simulated teeth 308 (i.e., crowns). Gingival cap 300 thus serves the aesthetic function of providing one or more simulated teeth, in addition to providing surface 302 for facilitating integration of the regenerative material with the ridge.

Reference is made to FIGS. 11A-F, which are schematic illustrations of several steps of a minimally-invasive alveolar ridge augmentation procedure that uses external gingival cap 300, as described hereinabove with reference to FIG. 10A and/or FIG. 10B, in accordance with an application of the present invention. The procedure is typically employed when a patient's maxillary or mandibular alveolar ridge 100 lacks sufficient bone width to support a dental implant, as shown in FIG. 11A. For example, the procedure may be employed for implanting an implant to replace the upper canines, lower molars, upper incisors, or lower incisors.

A surgeon begins the procedure by preparing the oral facial region, and administering a local anesthetic. A small incision is made through a site of gingiva 102, and a portion of the gingiva is loosened from the bone of ridge 100, thereby creating cavity 120 between the gingival portion and the bone, as shown in FIG. 11B. The gingiva is typically loosened by tunneling using a periosteal elevator (e.g., a Molt 9 periosteal elevator), as is known in the art. Alternatively, some configurations of mesher 200 may be used to loosen the gingiva.

As shown in FIG. 11C, external gingival cap 300 is affixed to the ridge such that surface 302 of the gingival cap that faces the ridge defines a desired geometry of the atrophied portion of the ridge. For some applications, the gingival cap is affixed to the ridge by snapping the cap around the posterior and anterior sides of the ridge, as shown in FIGS. 11C-E. For these applications, the gingival cap typically has the U-shape shown in FIG. 10A-B and FIGS. 11C-E. The gingival cap is held in place on the ridge by the pressure applied by one or more ridge-coupling portions of the cap. Alternatively or additionally, the gingival cap is affixed to the ridge using one or more fasteners 304, such as screws, tacks, or nails. In this latter case, the gingival cap does not necessarily cover portions of the ridge other than in a vicinity of cavity 120, and is thus not necessarily U-shaped.

For some applications, the gingival cap is shaped to a desired geometry appropriate for the specific patient, typically before affixing the cap to the ridge. For example, computer aided design/manufacturing (CAD/CAM) techniques known in the art for producing dental prostheses may be used. Alternatively or additionally, the dental surgeon shapes the gingival cap after affixing it to the ridge.

As shown in FIG. 11D, regenerative material 130 is introduced into the cavity between the gingiva and the ridge, either via the same incision used to loosen the gingiva, or via another opening through the gingiva. The regenerative material expands the cavity and pushes the gingival portion against surface 302 of the gingival cap, thereby causing the gingival portion and the cavity to assume the desired geometry defined by the gingival cap.

Alternatively, regenerative material 130 is introduced into the cavity before gingival cap 300 is affixed to the ridge. The subsequent placement of the gingival cap shapes the gingival portion and the injected material within the cavity.

As shown in FIG. 11E, the regenerative material is allowed to at least partially integrate with the bone of the ridge. Typically, one must wait several weeks for such integration to occur.

After waiting for the regenerative material to at least partially integrate with the bone (e.g., to fully integrate with the bone), the surgeon removes external gingival cap 300 from the ridge, as shown in FIG. 11F. A dental implant is typically implanted in the augmented ridge.

External gingival cap 300 generally protects the injection site without the need for the internal scaffolding provided by stent implant 10 or the bundle described hereinabove. Alternatively, the cap is used in combination with the stent implant or the bundle.

For some applications, external gingival cap 300 is used in combination with the cutting techniques described hereinabove with referenced to FIGS. 7-9. For some applications, gingival cap 300 is shaped so as to define one or more openings that are positioned and/or oriented to guide mesher 200 and/or a periosteal elevator to a desired location and/or with a desired orientation.

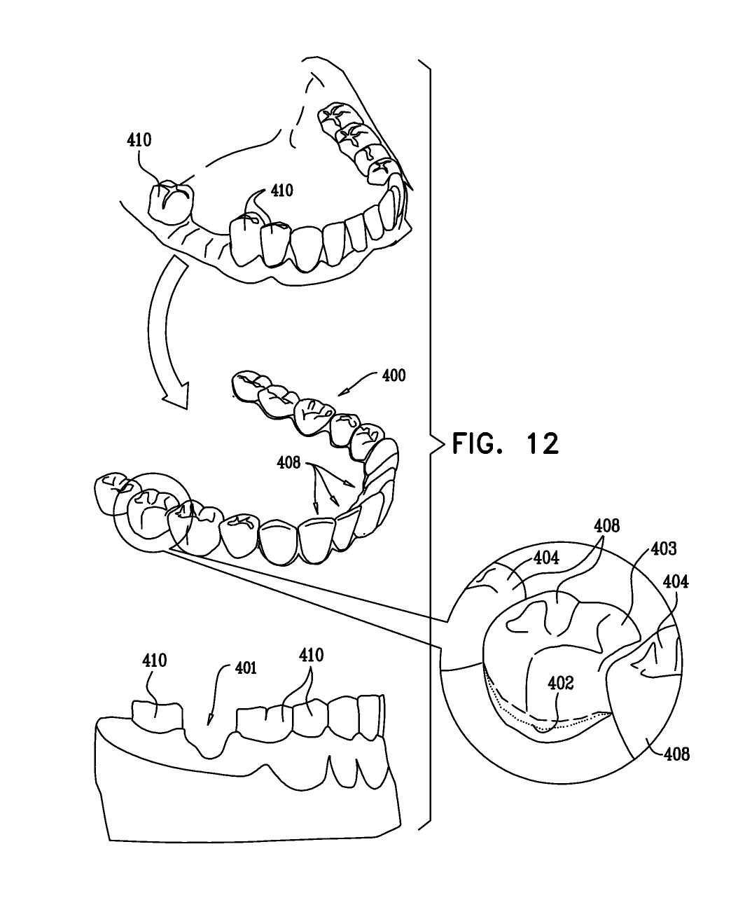

Reference is now made to FIG. 12, which is a schematic illustration of an external gingival cap 400, in accordance with an embodiment of the present invention. Like gingival cap 300, described hereinabove with reference to FIGS. 10A-B and 11A-F, gingival cap 400 is used for augmenting an atrophied portion 401 of alveolar ridge 100. Gingival cap 400 is shaped to define at least one cap portion 403, which is shaped so as to define a surface 402 that faces alveolar ridge 100 and defines a desired geometry of atrophied portion 401 of the alveolar ridge. Regenerative material introduced into cavity 120 of atrophied portion 401 between gingiva 102 and the atrophied ridge assumes the define geometry upon integrating with the bone of the ridge. In addition to providing the desired geometry, the gingival cap protects the regenerative material from moderate forces that are applied to the ridge during normal use of the mouth while the regenerative material integrates with the ridge. For some applications, the regenerative material comprises a fluid, a liquid, a gel, a particulate solid, or a suspension (i.e., a particulate solid suspended in a liquid).

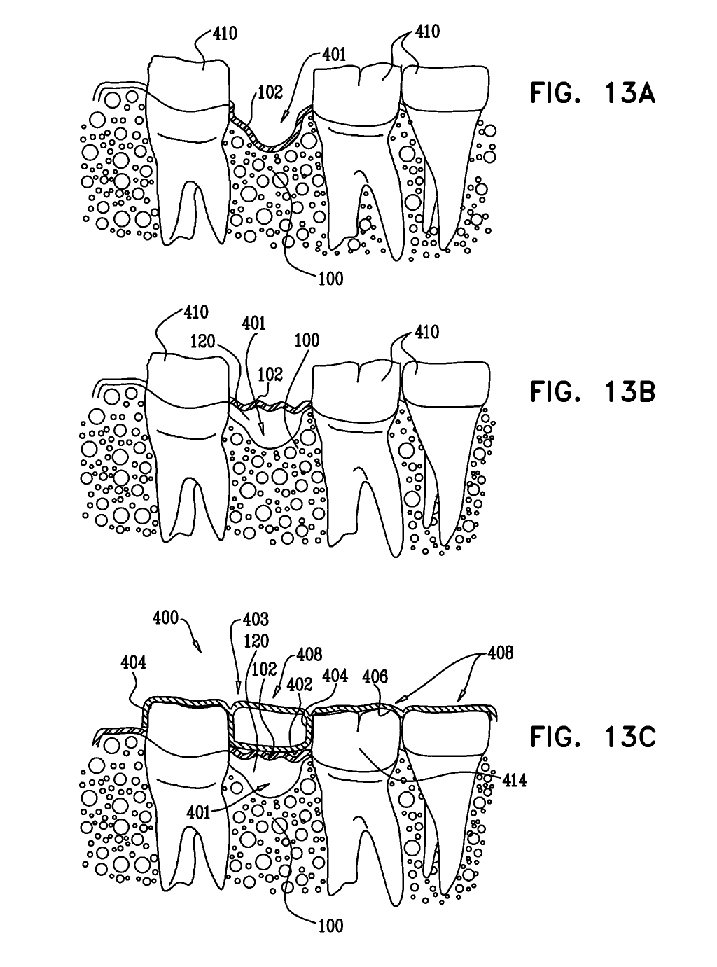

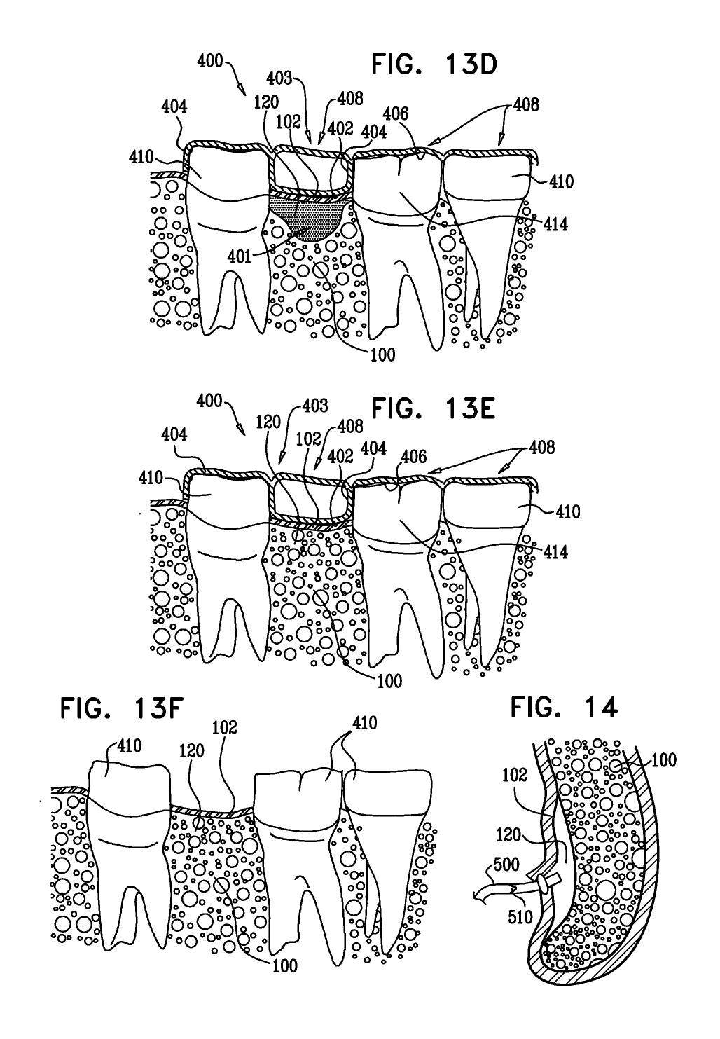

Gingival cap 400 additionally defines one or more (typically two or more) tooth-coupling portions 404, which are shaped to be removably coupled to respective teeth 410, typically in a vicinity of cavity 120, as described hereinbelow with reference to FIG. 13C. Teeth 410 may be natural teeth or prosthetic teeth implanted during an earlier dental procedure. Tooth-coupling portions 404 serve as ridge-coupling portions that couple gingival cap 400 to ridge 100 via teeth 410. For clarity of illustration, gingival cap 400 is shown in cross-section as contacting only occlusal, mesal, and distal surfaces of the teeth; the cap in addition typically contacts the buccal and lingual surfaces of the teeth.

Typically, gingival cap 400 is shaped such that surface 402 is positioned at atrophied portion 401 between two of tooth-coupling portions 404, such that the tooth-coupling portions hold surface 402 tightly in place after the gingival cap is coupled to the teeth. For some applications, gingival cap 400 is shaped so as to define a plurality of surfaces 402 that correspond to a plurality of cavities 120, either adjacent to one another, or interspersed between tooth-coupling portions 404.

Gingival cap 400 typically comprises a generally hard material, such as acetyl resin, methyl methacrylate, or acrylic. Typically, the cap has a thickness of between 0.1 mm and 10 mm. For some applications, the material of cap 400 has a memory, which allows the cap to flex while being placed over the wider portions of the teeth, and then return to its original shape in order to tightly grasp the teeth. As such, the gingival cap may be considered to snap onto the teeth.

For some applications, an outer surface of gingival cap 400 is shaped so as to define one or more simulated teeth 408 (i.e., crowns). Simulated teeth 108 are defined by: (a) one or more of tooth-coupling portions 404, (b) cap portion 403, or (c) both one or more of tooth-coupling portions 404 and cap portion 403. Gingival cap 400 thus serves the aesthetic function of providing one or more simulated teeth, in addition to providing surface 402 for facilitating integration of the regenerative material with the ridge. For some applications, gingival cap 400 is shaped so as to define an entire set of upper or lower simulate teeth 408, as shown in FIG. 12. Alternatively, the cap is shaped so as to define a partial set of one or more simulated teeth, typically two or more simulated teeth, or three or more simulated teeth. The inner surfaces of simulated teeth 408 defined by tooth-coupling portions 404 closely fit and conform to respective surfaces of the subject's teeth 410.

For some applications, gingival cap 400 provides simulated teeth 408 using at least a portion of the techniques described in U.S. Pat. No. 7,357,637 to Liechtung, which is incorporated herein by reference. These techniques are modified as described herein to provide the therapeutic functions of gingival cap 400. In contrast, the dental appliance described in the '637 patent appears to serve the purely aesthetic function of improving a patient's smile.

Reference is made to FIGS. 13A-F, which are schematic illustrations of several steps of a minimally-invasive alveolar ridge augmentation procedure that uses external gingival cap 400, in accordance with an application of the present invention. The procedure is typically employed when a patient's maxillary or mandibular alveolar ridge 100 lacks sufficient bone width to support a dental implant, as shown in FIG. 13A. For example, the procedure may be employed for implanting an implant to replace the upper canines, lower molars, upper incisors, or lower incisors.

A surgeon begins the procedure by preparing the oral facial region, and administering a local anesthetic. A small incision is made through a site of gingiva 102, and a portion of the gingiva is loosened from the bone of ridge 100, thereby creating cavity 120 between the gingival portion and the bone, as shown in FIG. 13B. The gingiva is typically loosened by tunneling using a periosteal elevator (e.g., a Molt 9 periosteal elevator), as is known in the art. Alternatively, some configurations of mesher 200, described hereinabove with referenced to FIGS. 7-9, may be used to loosen the gingiva.

As shown in FIG. 13C, external gingival cap 400 is affixed to the ridge such that surface 402 of the gingival cap that faces the ridge defines a desired geometry of the gingival port the atrophied portion of the ridge. Gingival cap 400 is affixed to the ridge by snapping tooth-coupling portions 404 to respective teeth 410 of the subject, which may be natural teeth or prosthetic teeth implanted during an earlier dental procedure. Typically, an interior surface 406 of each tooth-coupling portion 404 closely fits and conforms to the surface of a crown 414 of a corresponding tooth 410.

For some applications, gingival cap 400, including tooth-coupling portions 404, is custom fabricated for the individual patient being treated, typically before affixing the cap to the ridge. An impression of teeth 410 is made using well-known techniques, and the cap is fabricated using the impression, using techniques known in the art. Alternatively or additionally, computer aided design/manufacturing (CAD/CAM) techniques known in the art for producing dental prostheses may be used. Alternatively, the dental surgeon shapes the gingival cap after affixing it to the ridge. Further alternatively or additionally, the various sizes and configurations of the cap are provided, from which the surgeon selects the most appropriate fit.

Typically, gingival cap 400 is affixed to the ridge without using any adhesive, cement, or fasteners. Alternatively, the gingival cap is affixed using one or more fasteners, such as screws, tacks, or nails (configuration not shown), and/or a non-permanent adhesive.

As shown in FIG. 13D, regenerative material 130 is introduced into the cavity, either via the same incision used to loosen the gingiva, or via another opening through the gingiva. The bone graft expands the cavity and pushes the gingival portion against surface 402 of the gingival cap, thereby causing the gingival portion and the cavity to assume the desired geometry defined by the gingival cap.

Alternatively, regenerative material 130 is introduced into the cavity before gingival cap 400 is affixed to the ridge. The subsequent placement of the gingival cap shapes the gingival portion and the injected material within the cavity.

As shown in FIG. 13E, the regenerative material is allowed to at least partially integrate with the bone of the ridge. Typically, one must wait several weeks for such integration to occur.

After waiting for the regenerative material to at least partially integrate with the bone (e.g., to fully integrate with the bone), the surgeon removes external gingival cap 400 from the ridge, as shown in FIG. 13F. A dental implant is typically implanted in the augmented ridge.

External gingival cap 400 typically protects the injection site without the need for the internal scaffolding provided by stent implant 10 or the bundle described hereinabove. Alternatively, the cap is used in combination with the stent implant or the bundle.

For some applications, external gingival cap 400 is used in combination with the cutting techniques described hereinabove with referenced to FIGS. 7-9. For some applications, gingival cap 300 is shaped so as to define one or more openings that are positioned and/or oriented to guide mesher 200 and/or a periosteal elevator to a desired location and/or with a desired orientation

FIG. 14 is a schematic illustration a delivery tube 500 comprising a valve 510, for delivering regenerative material, in accordance with an application of the present invention. After an incision is made in gingiva 102, and the gingiva has been separated from alveolar ridge 100, such as described hereinabove with reference to FIGS. 6A-F, FIGS. 9, 11A-F, and/or 13A-F, delivery tube 500 is inserted into the incision, and sealingly held in place, such as using sutures or glue. Delivery tube 500 is used for delivery the regenerative material between gingiva 102 and alveolar ridge 100 (e.g., to cavity 120). Tube 500 comprises valve 510, which prevents outflow of the regenerative material once the material has been delivered.

For some applications, valve 510 is arranged in a fluid path defined by tube 500. For some applications, the valve is unidirectional, and is configured to allow passage of material through tube 500 toward cavity 120, and to prevent the passage in an opposite direction. Alternatively, the valve is bi-directional, and can be opened to allow passage of the material toward cavity 120, and subsequently closed to prevent the material from exiting. For some applications, the valve comprises a trap door valve, a faucet valve, a duckbill check valve, or a magnetic valve.

Further alternatively, the valve is coupled to an external surface of tube 500, and controllably applies pressure to the tube to block passage therethrough.

For some applications, tube 500 is used in combination with mesher 200, described hereinabove with reference to FIGS. 7-9. The tube is typically inserted after an incision has been made, the mesher has been used to cut the periosteum, and the gingiva has been stretched.

For some applications, an expandable chamber, such as a balloon, is provided for gradually separating the gingiva from the alveolar bone, thereby creating a cavity between the gingiva and the bone. An incision is made in the gingiva, and the expandable chamber is inserted through the incision. The expandable chamber is typically left in place for a period of several days to several weeks. The expandable chamber is configured to gradually automatically expand during this period, without being activated by any external mechanism. The expandable chamber is typically not connected to any external source of fluid or pressure, and is thus sealed from the external environment. The gradual expansion of the expandable chamber gently separates the gingiva from the bone.

For some applications, the expandable chamber gradually expands by absorbing biological fluid. For example, the expandable chamber may comprise a collagen sponge containing a hydroscopic material, such as algae. (It is known in the art to use algae as a cervical dilator.) Alternatively or additionally, the expandable chamber may comprise one or more springs therein that slowly expand the expandable chamber as the gingiva loosens. For some applications, the springs comprise a biodegradable material, such PGA-PLA.

For some applications, the expandable chamber is left in place temporarily until it sufficiently expands. The expandable chamber is removed during a subsequent surgical procedure. Bone regenerative material is inserted into the cavity made by the balloon, optionally using techniques described hereinabove.

For other applications, the expandable chamber is biodegradable, and contains a bone regenerative material. After expanding, the expandable chamber biodegrades, releasing the regenerative material into the newly created cavity.

For some applications, the balloon, in any of the configurations described above, is used in combination with other techniques described hereinabove. For example, the expandable chamber may be used in combination with the techniques described hereinabove with reference to FIGS. 1A-6F, FIGS. 7-9, FIGS. 10A-11F, FIGS. 12-13F, or FIG. 14.

For some applications, the techniques described hereinabove are practiced using an expandable chamber, such as a balloon, that is actively inflated during a procedure. For example, the expandable chamber may be inflated with a fluid (e.g., a liquid or a gas, such as air) provided by an external fluid source in fluid communication with the balloon. Alternatively, the expandable chamber may be inflated with regenerative material. For some applications, the expandable chamber is removed upon completion of the procedure, while for other applications, the expandable chamber is biodegradable and is left in place to biodegrade. For some applications, the volume and/or pressure of fluid (liquid or gas) or regenerative material is monitored as it is introduced into the expandable chamber, such as using a pressure gauge.

For some applications, the regenerative materials described herein may comprise one of the following commercially available fluid bone graft materials: DBX Paste (MTF), Allomatrix (Wright), Cerament (Bone Support), DynaGraft (Citagenix/ISOTIS), Fisiograft (Ghimas), Grafton (Osteotech), Optium DBM Gel (Lifenet/Depuy J&J), OsteoMax (Orthfix), PD VitalOs Cement (VitalOs), or Regenafil® (Exactech).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a gingival periosteum lining a bone, the apparatus comprising a periosteal mesher, which comprises:
a mesher surface; and
a plurality of cutting elements distributed over the mesher surface, which are configured to cut the gingival periosteum to increase flexibility thereof.

2. The apparatus according to claim 1, wherein the cutting elements comprise energy-applying elements, which are configured to cut the gingival periosteum by applying energy to the gingival periosteum.

3. The apparatus according to claim 2, wherein the energy-applying elements comprise electrodes.

4. The apparatus according to claim 3, further comprising a radiofrequency (RF) generator, which is configured to drive a RF current through the electrodes.

5. The apparatus according to claim 3, wherein the electrodes protrude from the mesher surface.

6. The apparatus according to claim 2, wherein the energy-applying elements comprise ultrasound transducers.

7. The apparatus according to claim 2, wherein the mesher comprises between 1 and 30 energy-applying elements.

8. (canceled)

9. The apparatus according to claim 1, wherein the cutting elements are configured to create cuts that extend through only a portion of a thickness of the gingival periosteum, and not entirely through the gingival periosteum.

10. The apparatus according to claim 1, wherein at least a portion of the cutting elements are arranged parallel to one another on the mesher surface.

11. The apparatus according to claim 1, further comprising a kit, which comprises the mesher and a bone regenerative material.

12. A method comprising:
inserting, between an alveolar ridge and gingiva lining the ridge, a periosteal mesher that includes a plurality of cutting elements distributed over a surface of the mesher; and
increasing flexibility of a gingival periosteum by cutting the gingival periosteum using the cutting elements.

13. The method according to claim 12, wherein the cutting elements include energy-applying elements, and wherein cutting the gingival periosteum comprises activating the energy-applying elements to apply energy to the gingival periosteum.

14. The method according to claim 13, wherein the energy-applying elements include electrodes, and wherein activating comprises driving a current through the electrodes.

15. The method according to claim 14, wherein driving the current comprises driving a radiofrequency (RF) current through the electrodes.

16. The method according to claim 13, wherein the energy-applying elements include ultrasound transducers, and wherein activating comprises activating the ultrasound transducers to generate ultrasound waves.

17. The method according to claim 12, wherein increasing the flexibility of the periosteum comprises facilitating stretching of the periosteum to encompass a larger volume between the periosteum and the ridge, and further comprising introducing a regenerative material between the periosteum and the ridge.

18. The method according to claim 17, wherein introducing comprises introducing the regenerative material via a delivery tube that includes a valve.

19. The method according to claim 12, wherein increasing the flexibility of the periosteum comprises stretching the cut gingival periosteum.

20. The method according to claim 12, wherein cutting comprises creating cuts that extend through only a portion of a thickness of the gingival periosteum, and not entirely through the gingival periosteum.

21-22. (canceled)

23. The method according to claim 12, further comprising:
before inserting, identifying that the gingiva is recessed; and
after cutting the gingival periosteum, stretching the gingiva toward crowns of one or more teeth of the ridge.

24. The method according to claim 12, wherein inserting comprises separating the gingiva from the ridge.

25. Apparatus for augmenting an atrophied portion of an alveolar ridge of a subject, the apparatus comprising an external gingival cap, which comprises:
at least one cap portion, which is shaped so as to define a surface that faces the alveolar ridge and defines a desired geometry of the atrophied portion of the ridge; and
one or more ridge-coupling portions, which are configured to be removably coupled to the alveolar ridge, and to hold the cap portion in place.

26. The apparatus according to claim 25, further comprising a kit, which comprises the external gingival cap and a bone regenerative material.

27. The apparatus according to claim 26, wherein the kit further comprises a delivery tube that comprises a valve, which delivery tube is configured to deliver the bone regenerative material.

28. The apparatus according to claim 25, wherein the external gingival cap further comprises one or more simulated teeth, coupled to the at least one cap portion.

29. The apparatus according to claim 25, wherein the one or more ridge-coupling portions comprise one or more tooth-coupling portions, which are configured to be removably coupled to respective ones of teeth of the ridge, in order to couple the external gingival cap to the alveolar ridge.

30. The apparatus according to claim 29, wherein the cap portion is shaped so as to define one or more simulated teeth.

31. The apparatus according to claim 29, wherein the one or more tooth-coupling portions are shaped so as to define one or more simulated teeth.

32. The apparatus according to claim 29, wherein the cap portion and the one or more tooth-coupling portions are shaped so as to define a plurality of simulated teeth.

33. A method for augmenting an alveolar ridge of a subject, comprising:
separating gingiva from an atrophied portion of the alveolar ridge to create a cavity between the gingiva and the atrophied portion of the ridge;
affixing an external gingival cap to the ridge such that a surface of a cap portion of the gingival cap that faces the ridge defines a desired geometry of the atrophied portion of the ridge;
before or after affixing the cap, introducing a regenerative material into the cavity;
waiting for the regenerative material to at least partially integrate with the bone of the ridge, such that the ridge assumes the desired geometry; and
after waiting, removing the external gingival cap from the ridge.

34. The method according to claim 33, further comprising, before separating the gingiva, making an incision through a site of the gingiva in a vicinity of the atrophied portion of the ridge.

35. The method according to claim 33, wherein introducing comprises introducing an amount of regenerative material sufficient to expand the cavity and push the gingiva against the surface of the gingival cap.

36. The method according to claim 33, wherein affixing comprises affixing one or more tooth-coupling portions of the gingival cap to respective teeth of the subject.

37. The method according to claim 36, wherein the cap portion is shaped so as to define one or more simulated teeth.

38. The method according to claim 36, wherein the one or more tooth-coupling portions are shaped so as to define one or more simulated teeth.

39. The method according to claim 36, wherein the cap portion and the one or more tooth-coupling portions are shaped so as to define a plurality of simulated teeth.

40. The method according to claim 33, wherein introducing comprises introducing the regenerative material via a delivery tube that includes a valve.

41-44. (canceled)
